(19) United States
(12) Patent Application Publication
(10) Pub. No.: US 2021/0369089 A1
(43) Pub. Date: Dec. 2, 2021

(54) ENDOSCOPE

(71) Applicant: FUJIFILM Corporation, Tokyo (JP)

(72) Inventors: Tsuneo FUKUZAWA, Ashigarakami-gun (JP); Yasuhiko MORIMOTO, Ashigarakami-gun (JP); Satoru OKADA, Ashigarakami-gun (JP)

(73) Assignee: FUJIFILM Corporation, Tokyo (JP)

(21) Appl. No.: 17/398,558

(22) Filed: Aug. 10, 2021

Related U.S. Application Data
(63) Continuation of application No. PCT/JP2020/009683, filed on Mar. 6, 2020.

(30) Foreign Application Priority Data
Mar. 7, 2019 (JP) 2019-041826

(51) Int. Cl. A61B 1/00; A61B 8/12

(52) U.S. Cl. CPC A61B 1/00098; A61B 8/12; A61B 1/00087; A61B 1/00147

(57) ABSTRACT

Provided is an endoscope that can fix a guidewire and stably hold a treatment tool. The endoscope includes a distal end body (36) having an elevator housing (62) that opens toward a first direction, a treatment tool lead-out port (80) that opens inside of the elevator housing (62), and an elevator (60) that is provided inside of the elevator housing (62) so as to be rotatable between an elevated position and a lowered position, the elevator (60) having a treatment tool guiding surface (60a). A facing wall portion (66), which is on a proximal end side and an opening part of the elevator housing (62), has a concave surface (96) that is formed on an inner side of the elevator housing (62) and a convex surface (94) that is formed at a position closer than the concave surface (96) to an opening. When the elevator (60) is in a forward position that is on the lowered position side relative to the elevated position, the concave surface (96) and the treatment tool guiding surface (60a) constitute a treatment tool holding portion (99) for holding a treatment tool, and, when the elevator (60) is in the elevated position, the convex surface (94) and the treatment tool guiding surface (60a) constitute a guidewire fixing portion (98) for fixing a guidewire.

### CROSS-REFERENCE TO RELATED APPLICATIONS

The present application is a Continuation of PCT International Application No. PCT/JP2020/009683 tiled on Mar. 6, 2020 claiming priority under 35 U.S.0 § 119(a) to Japanese Patent Application No. 2019-041826 filed on Mar. 7, 2019. Each of the above applications is hereby expressly incorporated by reference, in its entirety, into the present application.

### BACKGROUND OF THE INVENTION

### 1. Field of the Invention

The present invention relates to an endoscope and, in particular, to an endoscope including an elevator in a distal end body of an insertion section.

### 2. Description of the Related Art

Some existing endoscopes include an elevator and an elevator housing in a distal end body of an insertion section that is inserted into a body cavity. Such an endoscope can cause the elevator to elevate a treatment tool, which is inserted through a treatment tool insertion channel and is led out from an opening portion of the elevator housing, and can adjust the lead-out direction of the treatment tool by changing the elevation angle of the elevator (see for example, JP2017-086399A).

WO2018/079790A describes an endoscope in which, in order to suppress wobbling of a treatment tool, a second guide groove, which has an opening width that is smaller than the diameter of a treatment tool insertion hole, is formed in an inner surface of the treatment tool insertion hole.

### SUMMARY OF THE INVENTION

Endoscopes are used together with various treatment tools, such as puncture needles, guidewires, and stents. For example, the following treatment method has become widespread: a small hole is formed in a cyst by using a puncture needle, a guidewire is inserted through the small hole, a stem is placed in the cyst by using the guidewire as a guide, and thereby substances in the cyst are discharged to the alimentary canal.

It is practiced that, when pulling out a treatment tool (puncture needle) by using the guidewire as a guide, the guidewire is held between the elevator and the distal end body to increase the sliding resistance of the guidewire to suppress removal of the guidewire from the penetration position.

However, when pulling out the treatment tool, it often occurs that the guidewire moves together with puncture needle and the guidewire is unintentionally pulled out from the cyst. If the guidewire is removed, it is not possible to place the stent at a desirable position by using the guidewire as a guide after pulling out the puncture needle. Redoing placement of the guidewire is a factor that increases the operation time.

The endoscope described in WO2018/079790A, which holds a treatment tool by using the second guide groove and a first guide groove of the elevator in order to prevent wobbling of the treatment tool, cannot fix a guidewire having a smaller diameter than the treatment tool.

The present invention has been made in consideration of such circumstances, and an object thereof is to provide an endoscope that can fix a guidewire that is led out from a treatment tool lead-out port and that can stably hold a treatment tool without allowing a horizontal wobble of the treatment tool.

To achieve the object of the present invention, an endoscope according to the present invention includes a distal end body that is provided at a distal end of an insertion section that extends in a longitudinal axis direction. The distal end body has an elevator housing that opens toward a first direction that is perpendicular to the longitudinal axis direction, a treatment tool lead-out port that opens inside of the elevator housing, and an elevator that is provided inside of the elevator housing so as to be rotatable between an elevated position and a lowered position, the elevator having a treatment tool guiding surface. The elevator housing has a facing wall portion in a part that is on a proximal end side in the longitudinal axis direction and that is an opening part of the elevator housing. The facing wall portion has a concave surface that is formed on an inner side of the elevator housing and a convex surface that is formed at a position closer than the concave surface to an opening. When the elevator is in a forward position that is on the lowered position side relative to the elevated position, the concave surface and the treatment tool guiding surface constitute a treatment tool holding portion for holding a treatment tool, and, when the elevator is in the elevated position, the convex surface and the treatment tool guiding surface constitute a guidewire fixing portion for fixing a guidewire.

With the endoscope according to the present invention, the facing wall portion, which is disposed in the opening part of the elevator housing, has the convex surface and the concave surface; it is possible to fix a guidewire by constituting the guidewire fixing portion by the convex surface and the treatment tool guiding surface of the elevator; and it is possible to hold a treatment tool by constituting the treatment tool holding portion by the concave surface and the treatment tool guiding surface. Accordingly, it is possible to perform both of fixing of the guidewire and stable holding of the treatment tool.

### BRIEF DESCRIPTION OF THE DRAWINGS

FIG. 1 is an overall view of an ultrasonic endoscope according to the present invention ;

FIG. 2 is an external perspective view of a distal end portion when an elevator is in a lowered state;

FIG. 3 is an external perspective view of the distal end portion when the elevator is in an elevated state;

FIG. 4 is a side sectional view of the distal end portion;

FIG. 5 is a side sectional view illustrating a state in which a guidewire is fixed;

FIG. 6 is a view as seen from the direction C of FIG. 5;

FIG. 7 is a side sectional view illustrating a state in which a treatment tool is held;

FIG. 8 is a view as seen from the direction D of FIG. 7; and

FIG. 9 is side sectional view illustrating the configuration of a distal end portion of a second embodiment.

### DESCRIPTION OF THE PREFERRED EMBODIMENTS

Hereafter, an endoscope according to the present invention will be described with reference to the drawings.

### Endoscope

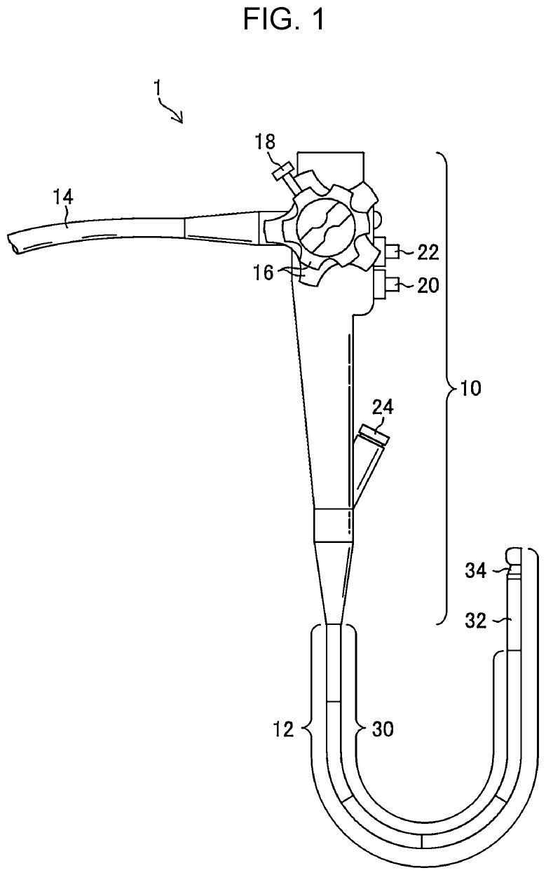

FIG. 1 is an overall view of an endoscope I to which the present invention is applied. Although an ultrasonic endoscope will be described as an example in the embodiment, the present invention can be applied also to an endoscope other than an ultrasonic endoscope. That is, the present invention can be applied to any endoscope that has an elevator and an opening for leading out a treatment tool.

The endoscope 1 in the figure is constituted by an operation section 10 that an operator grips to perform various operations, an insertion section 12 that is inserted into a body cavity of a patient, and a universal cord 14. The endoscope 1 is connected, via the universal cord 14, to system component devices (not shown), such as a processor device and a light source device, that constitute an endoscope system.

Various operation members, which are operated by an operator, are provided in the operation section 10. For example, an angle knob 16, an elevating operation lever 18, an air/water supply button 20, a suction button 22, and the like are provided.

In the operation section 10, a treatment tool insertion port 24, for inserting a treatment tool into a treatment tool insertion channel that extends through the insertion section 12, is provided.

The insertion section 12 extends out from the distal end of the operation section 10, and the entirety of the insertion section 12 has a small-diameter elongated shape.

The insertion section 12 is constituted by a flexible portion 30, a bending portion 32, and a distal end portion 34, sequentially from the proximal end side toward the distal end side.

The flexible portion 30 occupies most part of the insertion section 12 from the proximal end side, and has flexibility of bending in any directions. When the insertion section 12 is inserted into a body cavity, the flexible portion 30 bends along an insertion path into the body cavity.

By rotating the angle knob 16 of the operation section 10, the bending portion 32 can be bent in the up-down direction and in the left-right direction. By bending the bending portion 32, the distal end portion 34 can be directed in a desirable direction,

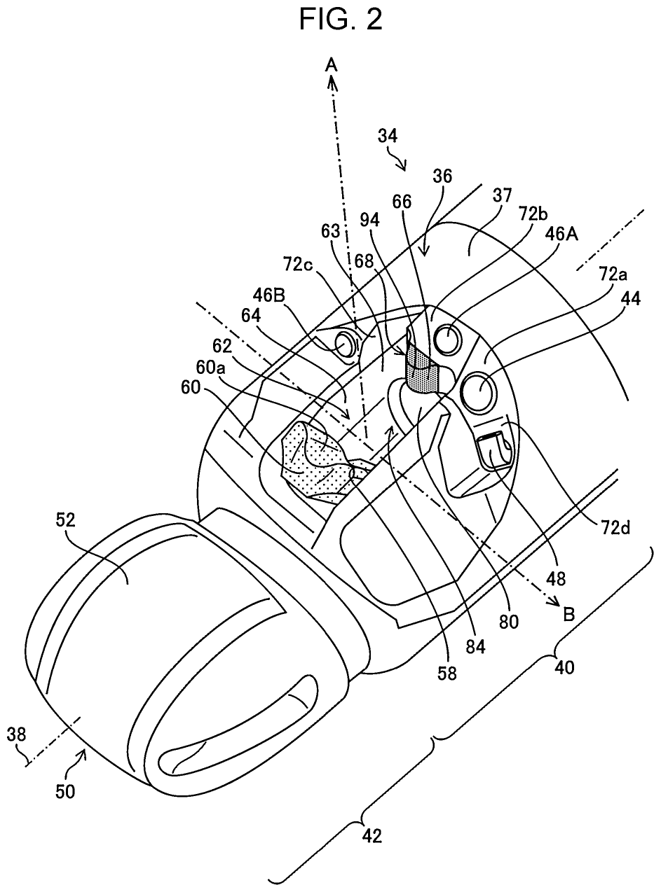

The distal end portion 34 includes a distal end body 36. Which will be described below in detail with reference to FIGS. 2 to 4. An ultrasonic transducer 50, which has a plurality of ultrasonic vibrators, is provided on the distal end side of the distal end body 36.

The universal cord 14 illustrated in FIG. 1 contains an electric cable, a light guide, and a fluid tube. A connector is included in an end portion (not shown) of the universal cord 14. By connecting the connector to predetermined system component devices such as a processor device and a light source device, electric power, a control signal, illumination light, a liquid, and a gas, and the like, which are necessary for operating the endoscope 1, are supplied from the system component devices to the endoscope 1. Data of an observation image captured by an imaging unit and data of an ultrasound image obtained by the ultrasonic transducer are transmitted from the endoscope 1 to the system component devices. The observation image and the ultrasound image transmitted to the system component devices are displayed on a monitor, and an operator and the like can observe these images.

### First Embodiment

### Configuration of Distal End Portion

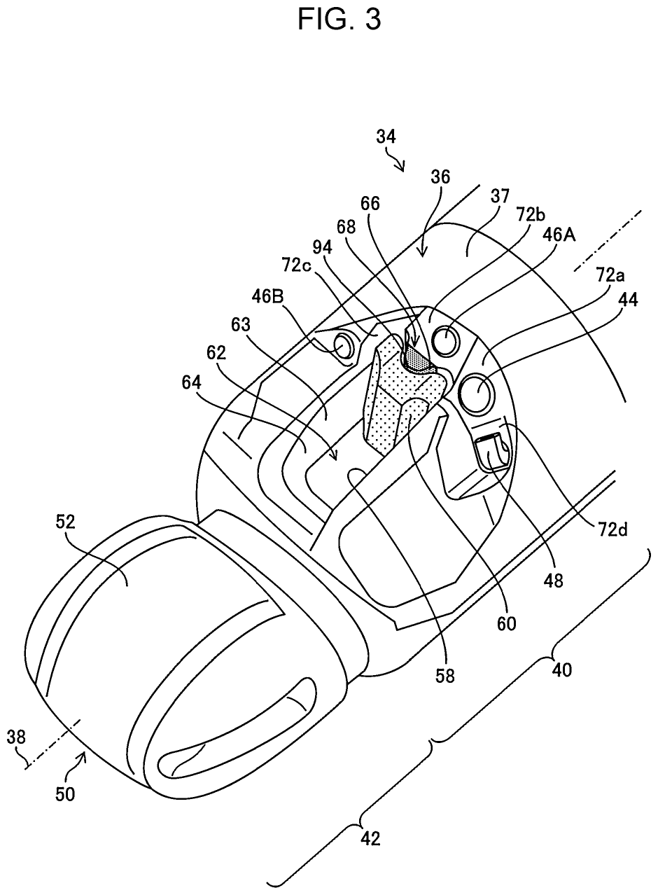

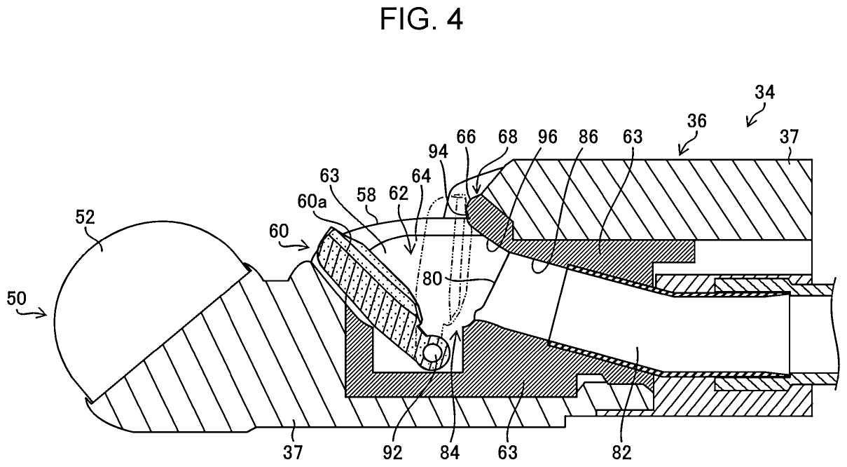

Next, the configuration of the distal end portion 34 of the insertion section 12 of the endoscope of the first embodiment will be described. FIG. 2 is an external perspective view of the distal end portion 34, illustrating a state in which an elevator 60 is in a lowered position. FIG. 3 is an external perspective view of the distal end portion 34, illustrating a state in which the elevator 60 is in an elevated position. FIG. 4 is a side sectional view,

The distal end portion 34 has the distal end body 36 that forms an outer wall and an inner partition wall thereof Each component disposed in the distal end body 36 is accommodated and held in a housing portion included in the distal end body 36,

Although details are omitted, a part of the distal end body 36 is removable as a separate block, and each component can be mounted in a predetermined housing portion in a state in which the separate block is removed. After each component has been mounted in the housing portion, the separate block is attached to the distal end body 36, and thereby each component is accommodated and held in the housing portion and fixed to the distal end portion 34.

The distal end body 36 is made of an insulating material having insulation ability, the examples of which are plastic resin materials such as a methacrylate resin, a polyphenylsulfone resin, a polyetherimide resin, a polvetheretherketone resin, and polycarbonate.

As illustrated in FIGS. 2 to 4, the distal end body 36 is constituted by: a base portion 40 in which an observation optical system, a treatment tool lead-out portion, an elevator that guides a treatment tool led out from a treatment tool lead-out portion, and the like are formed; and an extension portion 42 that extends from the base portion 40 toward the distal end side and that holds the ultrasonic transducer 50.

In the extension portion 42, the ultrasonic transducer 50, which is of a convex type and which transmits and receives ultrasound, is disposed. The ultrasonic transducer 50 has an ultrasound transmitting/receiving surface 52, and the ultrasound transmitting/receiving surface 52 is formed by arranging ultrasonic vibrators in a curved shape in a longitudinal axis 38 direction of the insertion section 12. The ultrasonic transducer 50 obtains data for generating an ultrasound image of a body tissue.

As illustrated in FIG. 2 and FIG. 3, in the distal end body 36, an observation window 44, a first illumination window 46A, a second illumination window 46B, an air/water supply nozzle 48, and an opening portion 58 for leading out a treatment tool are provided.

The opening portion 58 is provided in the base portion 40 of the distal end body 36, and a treatment tool is led out from the opening portion 58 to the scanning range of ultrasound of the ultrasonic transducer 50. The periphery of an elevator housing 62 is formed by an elevator housing forming wall 64, and the opening portion 58 is formed so as to open toward a first direction that is perpendicular the longitudinal axis 38 direction of the insertion section 12 of the elevator housing 62.

As illustrated in FIGS. 2 and 4, a treatment tool lead-out portion 84, which has a treatment tool lead-out port 80 that opens inside of the elevator housing 62, is disposed on the proximal end side of the elevator housing 62. The treatment tool lead-out port 80 communicates with the treatment tool insertion port 24 of the operation section 10 (see Fig.) via a treatment tool insertion channel 82, which is disposed so as to extend through the insertion section 12. Thus, when the endoscope is inserted into a body cavity to perform treatment or observation, a treatment tool inserted from the treatment tool insertion port 24 is led out from the treatment tool lead-out port 80 (see FIG. 4) to the elevator housing 62,

The elevator 60 is disposed in the elevator housing 62 at a position in front of the treatment tool lead-out port 80. The elevator 60 is rotatable between an elevated position and a lowered position around a rotational axis 92. The elevator 60 is made of a metal material such as a stainless steel, and has a treatment tool guiding surface 60a, which has a concave shape that is upwardly curved from the proximal end side toward the distal end side of the distal end body 36, on the upper surface side thereof. A treatment tool led out from the treatment tool lead-out port 80 is guided along the treatment tool guiding surface 60a upward with respect to the longitudinal axis direction of the insertion section 12, and is led out from the opening portion 58 on the upper side of the elevator housing 62.

By operating the elevating operation lever 18 illustrated in FIG. 1, the elevator 60 rotates around the rotational axis 92 to perform an elevating motion. The lead-out direction (lead-out angle) of the treatment tool, which is led out from the opening portion 58, can be changed by causing the elevator 60 to perform an elevating motion and by adjusting the elevation angle from a lowered state.

The distal end body 36 has an elevation unit 63, and the elevator 60 is disposed in the elevation unit 63. The elevation unit 63 is made of, for example, an anti-corrosive metal material

The treatment tool insertion channel 82 illustrated in FIG. 4 is connected also to a suction channel (not shown). By operating the suction button 22 shown in FIG. 1, a body fluid and the like can be sucked from the treatment tool lead-out port 80 via the opening portion 58.

The observation window 44 is disposed in an observation means forming surface 72a provided on the proximal end side of the elevator housing 62. An imaging system unit, in which an image-forming optical system and a solid-state imaging element which constitute an imaging unit are integrally assembled, is accommodated inside of the observation window 44, Thus, when light from a region to be treated, which is in the field of view of the imaging unit, is taken in through the observation window 44, the light passes through the image-forming optical system and forms an observation image in the solid-state imaging element. That is, an image of the region to be treated is captured by the solid-state imaging element.

The first illumination window 46A and the second illumination window 46B are provided in illumination means forming surfaces 72b and 72c. A light emission portion that constitutes an illumination unit is accommodated inside of each of the first illumination window 46A and the second illumination window 46B. From the light emission portion, illuminan on light that is transmitted via a light guide from the light source device connected to the universal cord 14 is emitted. A region to be treated, which is in the field of view of the imaging unit, is irradiated with the illumination light via the first illumination window 46A and the second illumination window 46B.

The air/water supply nozzle 48 is provided in a nozzle forming surface 72d. By operating the air/water supply button 20 shown in FIG. 1, a cleaning liquid, water, air, or the like is ejected toward the observation window 44 from the air/water supply nozzle 48 shown in FIGS. 2 and 3, and cleaning of the observation window 44 and the like are performed.

Next, the positional relationship among the opening portion 58, the elevator housing 62, and the observation window 44 will be described. As illustrated in FIG. 2, the position of the observation window 44 in the first direction indicated by an arrow A (direction in which the opening portion 58 opens) is located on a side opposite to the elevator housing 62 when the position of the opening portion 58 is defined as a reference position. That is, when the distal end body 36 is projected onto an imaginary plane perpendicular to the longitudinal axis 38 direction, the observation window 44 is disposed on the opening side (the opening portion 58 side) of the elevator housing 62. By thus positioning the observation window 44 above the opening portion 58, at a position where a treatment tool is led out from the opening portion 58, it is possible bring the treatment tool into the observation field of view of the observation window 44. Accordingly, it is possible to guide the treatment tool to a target position and to improve the accuracy in guiding the treatment tool to the target position.

Regarding the positions of the observation window 44 and the elevator housing 62 in a second direction indicated by an arrow B in FIG. 2, preferably, the observation window 44 is disposed so as to be offset from the elevator housing 62 in the direction indicated by the arrow B. The expression “the observation window 44 is disposed so as to be offset from the elevator housing 62 in the direction indicated by the arrow B” means that the center line of the observation window 44 is displaced in the direction indicated by the arrow B with respect to the center line of the elevator 60. With such a configuration, even in a state in which the elevator 60 is elevated and the treatment tool is led out from the opening portion 58, it is possible to prevent the observation field of view of the observation window 44 from being blocked by the treatment tool and the elevator 60, and it is possible to reliably identify the treatment position through the observation window 44.

The distal end body 36 is formed by mounting the elevation unit 63 made of a metal into a body case 37 made of a resin. A part of the elevator housing 62 is constituted by the body case 37, and the other part of the elevator housing 62 is constituted by the elevation unit 63.

The treatment tool lead-out port 80, which opens inside of the elevator housing 62, is provided in the elevation unit 63. The treatment tool lead-out port 80 is formed by a lead-out port forming wall 86 provided therearound, and is connected to the distal end of the treatment tool insertion channel 82. That is, the distal end of the treatment tool insertion channel 82 is connected to the elevation unit 63 and a treatment tool that is inserted through the inside of the treatment tool insertion channel 82 passes through the lead-out port forming wall 86 and the treatment tool lead-out port 80 and is led out to the elevator housing 62.

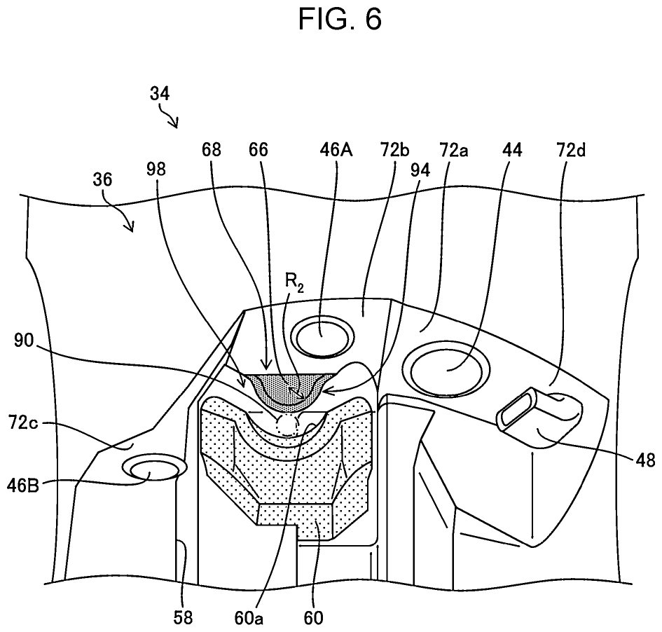

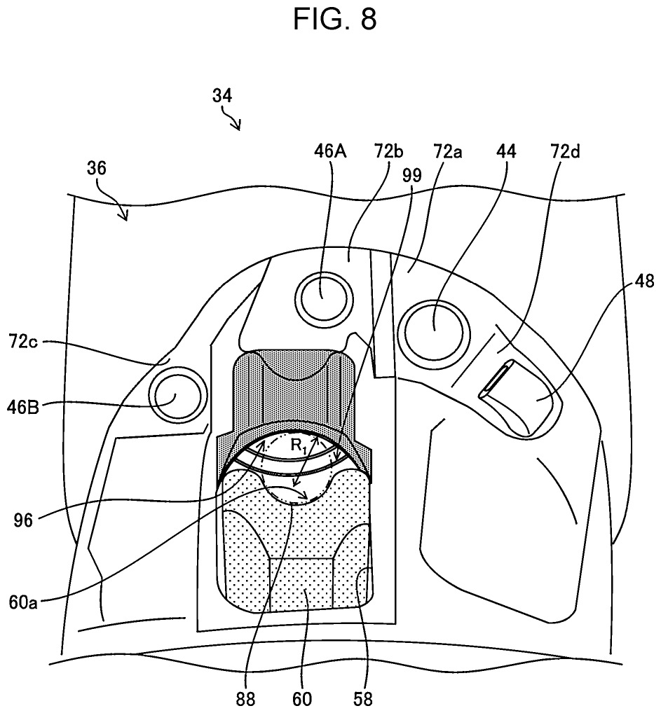

The elevator housing forming wall 64, which is on the proximal end side in the longitudinal axis direction of the elevator housing 62, has a facing wall portion 66 in a part of the opening portion 58. The facing wall portion 66 has a convex portion 68 that protrudes toward the distal end side in the longitudinal axis 38 direction. The convex portion 68 has a concave surface 96 (see FIG. 8) that is formed on the inner side of the elevator housing 62 and a convex surface 94 (see FIG. 6) that is formed at a position closer than the concave surface 96 to the opening. In a state in which the elevator 60 is in an elevated position as shown by a two-dot chain line in FIG. 4, the convex surface 94 is provided at a position such that the convex surface 94 faces the treatment tool guiding surface 60a of the elevator 60.

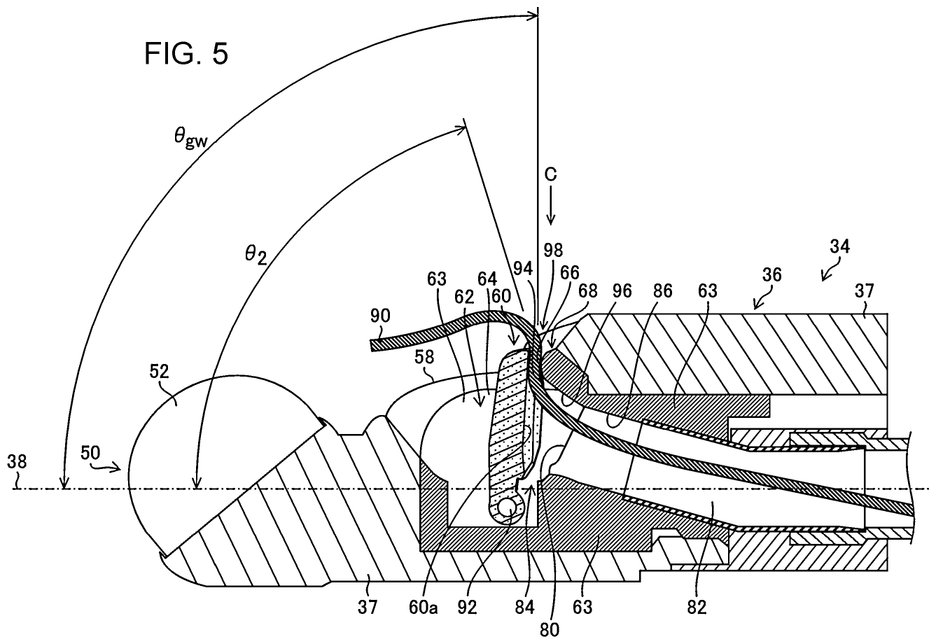

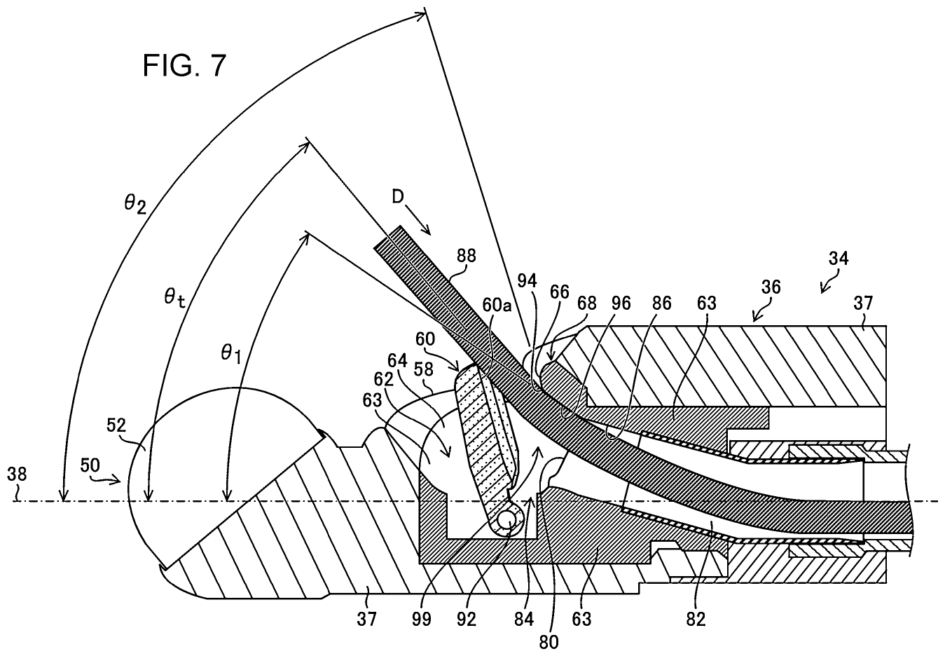

FIGS. 5 to 8 illustrate a state in which a treatment tool or a guidewire is held by the facing wall portion 66 and the elevator 60. FIG. 5 is a side sectional view illustrating a state in which a guidewire 90 is fixed by the elevator 60 and the convex surface 94, and FIG. 6 is a view as seen from the direction C of FIG. 5. FIG. 7 is a side sectional view illustrating a state in which a treatment tool 88 is held by the elevator 60 and the concave surface 96, and FIG. 8 is a view as seen from the direction I) of FIG. 7. In FIGS. 6 and 8, for ease of understanding the structure of the distal end portion 34, the guidewire 90 and the treatment tool 88 are shown by two-dot chain lines.

As illustrated in FIGS. 5 and 6, the convex surface 94 has an arc shape along the concave shape of the treatment tool guiding surface 60a and has a convex shape that protrudes in a direction toward the distal end side in the longitudinal axis 38 direction. In a state in which the elevator 60 is in the elevated position, the treatment tool guiding surface 60a and the convex surface 94 face each other, and thereby the treatment tool guiding surface 60a and the convex surface 94 are positioned close to each other to form a fixing space in which the guidewire 90 is to be fixed. That is, the treatment tool guiding surface 60a and the convex surface 94 constitute a guidewire fixing portion 98 that fixes the guidewire 90, in FIG. 6, the treatment tool guiding surface 60a has an arc shape, and the fixing space has a U-shape. However, the shapes are not limited to these. By positioning the treatment tool guiding surface 60a and the convex surface 94 close to each other, even if the guidewire 90 is displaced in a horizontal direction in FIG. 6 (direction B in FIG. 2), it is possible to grip the guidewire 90 at a certain position in the U-shaped fixing space, and to fix the guidewire 90 by using the guidewire fixing portion 98. Moreover, by locating the treatment tool guiding surface 60a and the convex surface 94 close to each other, it is possible to hold a guidewire 90 having a small diameter between the treatment tool guiding surface 60a and the convex surface 94 and to lock the guidewire 90. The expression “protrudes in a direction toward the distal end side” means that the protruding direction of the convex surface has a component that is directed toward the distal end, and also includes the meaning that the protruding direction of the convex surface is displaced from the distal end side in the first direction (the up-down direction in FIG. 5).

in a state in which the elevator 60 is in the elevated position, the width of the gap between the treatment tool guiding surface 60a and the convex surface 94 is preferably less than or equal to 0.5 mm. Because the diameter of a general guidewire is 0.6 mm, by making the width of the gap less than or equal to 0.5 mm, it is possible to hold the guidewire between the convex portion 68 and the treatment tool guiding surface 60a.

The concave surface 96 is formed on the elevator housing 62 side of the convex portion 68 of the facing wall portion 66. As illustrated in FIGS. 7 and 8, the concave surface 96 has an arc shape that is concave in such a way the concave surface 96 and the treatment tool guiding surface 60a, which is concave, are separated from each other. In a state in which the elevator 60 is in a forward position that is on the lowered position side relative to the elevated. position, the treatment tool guiding surface 60a and the concave surface 96 form a holding space in which the treatment tool 88 is to be held. That is, the treatment tool guiding surface 60a and the concave surface 96 constitute a treatment tool holding portion 99 for holding the treatment tool 88. By separating the treatment tool guiding surface 60a and the concave surface 96 from each other, it is possible to hold a treatment tool 88 having a larger diameter than the guidewire 90 by using the treatment tool guiding surface 60a and the concave surface 96, and it is possible to stably hold the treatment tool without allowing the treatment tool 88 to be displaced in a horizontal direction in FIG. 7 (direction B in FIG. 2).

The expression “a state in which the elevator is in a forward position that is on the lowered position side relative to the elevated position” means the maximally elevated position when the treatment tool 88 is held by the treatment tool guiding surface 60a. It may not be possible to move the elevator 60 to the elevated position in a state in which the treatment tool 88 is held, because the treatment tool 88 has a larger diameter and a higher bending stiffness than the guidewire 90. It is possible to move the elevator 60 to the elevated position in a state in which the treatment tool 88 is held, because the guidewire 90 has a smaller diameter and a lower bending stiffness than the treatment tool 88.

Relationship among Convex Surface, Concave Surface, and Lead-Out Direction of Treatment Tool

Referring to FIGS. 5 and 7, the relationships among the angles between the convex surface 94, the concave surface 96, and the lead-out direction of the treatment tool and the distal end direction of the longitudinal axis 38 direction will be described.

As illustrated in FIG. 7, when the facing wall portion 66 is seen from the second direction that is perpendicular to the longitudinal axis 38 direction of the distal end portion 34 and that is perpendicular to the first direction (the up-down direction in FIGS. 5 and 7), preferably, the following expression (1) is satisfied, where θ₁ is the angle between the concave surface 96 and the distal end direction of the longitudinal axis 38 direction, and 0: is the angle between the convex surface 94 and the distal end direction of the longitudinal axis 38 direction.

θ₁<θ₂ (1)

As illustrated in Pigs. 5 and 7, preferably, the following expressions (2) and (3) are satisfied, where θₜ is the angle between the lead-out direction of the treatment tool 88 and the distal end direction of the longitudinal axis 38 direction when the elevator 60 is in the forward position that is on the lowered position side relative to the elevated position, and θ_{gw} is the angle between the lead-out direction of the guidewire 90 and the distal end direction of the longitudinal axis 38 direction when the elevator 60 is in the elevated position.

θ₁≤θₜ<θ₂ (2)

θ₂≤θ_{gw} (3)

The lead-out direction of the treatment tool 88 led out from the treatment tool lead-out port 80 is adjusted by the elevator 60. When θ₁≤θₜ is satisfied, it is possible to bring the treatment tool 88 into contact with the concave surface 96 to form a fulcrum for changing the lead-out direction. Thus, it is possible to stably hold the treatment tool 88 by using the treatment tool guiding surface 60a and the concave surface 96.

When θₜ<θ₂ is satisfied, it is possible to prevent the treatment tool 88 from coming into contact with the convex surface 94 and to prevent the lead-out direction of the treatment tool 88 from being changed. When θ₂≤θ_{gw} is satisfied, it is possible to bring the guidewire 90 led out from the opening portion 58 into contact with the convex surface 94 and to reliably fix the guidewire 90 by using the treatment tool guiding surface 60a and the convex surface 94.

Preferably, the angle θ_{gw} is less than or equal to 90°. When the angle is less than or equal to 90°, in an operation of pulling out the treatment tool while leaving the guidewire, when the treatment tool 88 is pulled out, it is possible to reduce the reaction (movement) of the guidewire 90 before the guidewire 90 is fixed by the guidewire fixing portion 98. Thus, it is possible to prevent the guidewire 90 from being removed from the penetration point.

Referring back to FIGS. 6 and 8, preferably, the radius of curvature R₁ of the concave surface 96 is greater than the radius of curvature R₂ of the convex surface 94. As described above, the convex surface 94 is configured to be capable of becoming close to the treatment tool guiding surface 60a in order to form a U-shaped fixing space between the convex surface 94 and the treatment tool guiding surface 60a. Accordingly, preferably, the radius of curvature R2 of the convex surface 94 is small so that the convex surface 94 can enter the concave portion of the treatment tool guiding surface 60a having a concave shape. The concave surface 96 and the treatment tool guiding surface 60a constitute the treatment tool holding portion 99 and hold the treatment tool 88, which has a greater diameter than the guidewire 90. Accordingly, it is possible to make it easier to hold the treatment tool 88 by increasing the radius of curvature R₁.

As described above, with the present embodiment, the facing wall portion 66 of the elevator housing 62 has the convex surface 94 provided at the opening position of the elevator housing 62 and the concave surface 96 provided on the inner side of the elevator housing 62 relative to the convex surface 94, the concave surface 96 and the treatment tool guiding surface 60a constitute the treatment tool holding portion 99, and the convex surface 94 and the treatment tool guiding surface 60a constitute the guidewire fixing portion 98. Thus, it is possible to reliably fix the guidewire 90 by using the guidewire fixing portion 98, and, by holding the treatment tool 88 by using the treatment tool holding portion 99, it is possible to stably lead out the treatment tool 88 from the opening portion 58 without allowing a horizontal wobble.

### Second Embodiment

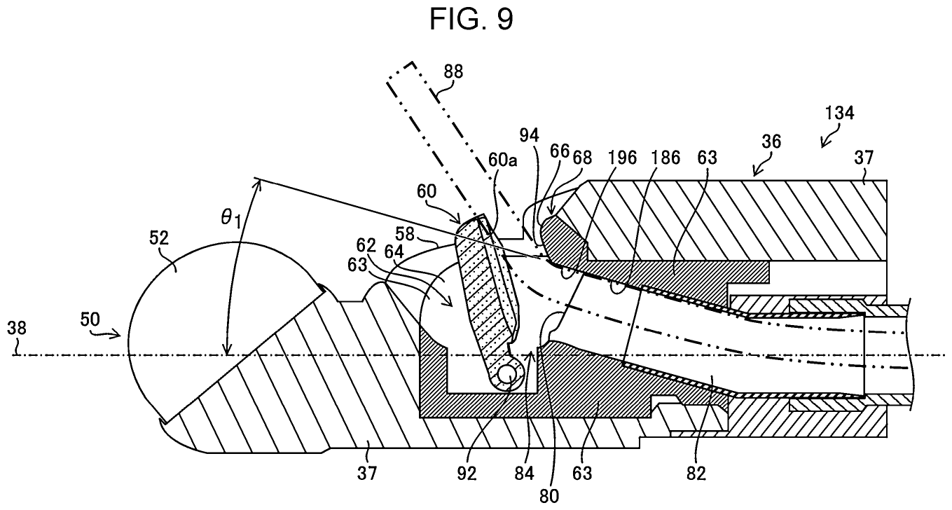

FIG. 9 is a side sectional view illustrating the configuration of a distal end portion 134 of an endoscope of a second embodiment. In the distal end portion 34 of the first embodiment, the angle between the lead-out port forming wall 86 and the distal end in the longitudinal axis 38 direction and the angle between the concave surface 96 and the distal end in the longitudinal axis 38 direction are different angles. In contrast, in the distal end portion 134 of the second embodiment, the angle formed between a lead-out port forming wall 186 and a line toward the distal end (that is, distal end direction) in the longitudinal axis 38 direction and the angle between a concave surface 196 and the distal end in the longitudinal axis 38 direction are the same angle, and the lead-out port forming wall 186 and the concave surface 196 are integrally formed.

Also when the lead-out port forming wall 186 and the concave surface 196 are integrally formed at the same angle, it is possible to hold the treatment tool by using the concave surface 196 and the treatment tool guiding surface 60a, to prevent a horizontal wobble of the treatment tool, and to stably hold the treatment tool,

Also in the distal end portion 134 of the endoscope of the second embodiment, when the angle θ₁ between the concave surface 196 and the distal end direction of the longitudinal axis 38 direction satisfies the conditions of the expressions (1) to (3) described above, advantageous effects that are the same as those of the endoscope of the first embodiment can be obtained.

In the foregoing description, a convex-type ultrasonic transducer has been described, However, the present invention is not limited to a convex-type ultrasonic transducer, and can be applied also to a radial-type ultrasonic transducer.

### REFERENCE SIGNS LIST

1 endoscope

10 operation section

12 insertion section

14 universal cord

16 angle knob

18 elevating operation lever

20 air/water supply button

22 suction button

24 treatment tool insertion port

30 flexible portion

32 bending portion

34, 134 distal end portion

36 distal end body

37 body case

38 longitudinal axis of insertion section

40 base portion

42 extension portion

44 observation window

46A first illumination window

46B second illumination window

48 air/water supply nozzle

50 ultrasonic transducer

52 ultrasound transmitting/receiving surface

58 opening portion

60 elevator

60a treatment tool guiding surface

62 elevator housing

63 elevation unit

64 elevator housing forming wall

66 facing wall portion

68 convex portion

72a observation means forming surface

72b, 72c illumination means forming; surface

72d nozzle forming surface

80 treatment tool lead-out port

82 treatment tool insertion channel

84 treatment tool lead-out portion

86, 186 lead-out port forming wall

88 treatment tool

90 guidewire

92 rotation axis

94 convex surface

96, 196 concave surface

98 guidewire fixing portion

99 treatment tool holding portion

What is claimed is:

1. An endoscope comprising:
a distal end body that is provided at a distal end of an insertion section that extends in a longitudinal axis direction,
wherein the distal end body has
an elevator housing that opens toward a first direction that is perpendicular to the longitudinal axis direction,
a treatment tool lead-out port that opens inside of the elevator housing, and
an elevator that is provided inside of the elevator housing so as to be rotatable between an elevated position and a lowered position, the elevator having a treatment tool guiding surface,
wherein the elevator housing has a facing wall portion in a part that is on a proximal end side in the longitudinal axis direction and that is an opening part of the elevator housing,
wherein the facing wall portion has a concave surface that is formed on an inner side of the elevator housing and a convex surface that is formed at a position closer than the concave surface to an opening, and
wherein, when the elevator is in a forward position that is on the lowered position side relative to the elevated position, the concave surface and the treatment tool guiding surface constitute a treatment tool holding portion for holding a treatment tool, and, when the elevator is in the elevated position, the convex surface and the treatment tool guiding surface constitute a guidewire fixing portion for fixing a guidewire.

2. The endoscope according to claim 1,
wherein the treatment tool guiding surface has a concave shape,
wherein the treatment tool holding portion has a holding space in which the treatment tool is to be held, the holding space being formed between the treatment tool guiding surface and the concave surface in such a way that the treatment tool guiding surface and the concave surface are separated from each other, and
wherein the guidewire fixing portion has a fixing space in which the guidewire is to he fixed, the fixing space being formed between the treatment tool guiding surface and the convex surface in such a way that the treatment tool guiding surface and the convex surface are close to each other.

3. The endoscope according to claim 1,
wherein, when the facing wall portion is seen from a second direction that is perpendicular to the longitudinal axis direction and that is perpendicular to the first direction, the following expression (1) is satisfied:
θ₁<θ₂ (1),
where θ₁ is an angle between the concave surface and a distal end direction of the longitudinal axis direction, and θ₁ is an angle between the convex surface and the distal end direction of the longitudinal axis direction.

4. The endoscope according to claim 3,
wherein the following expression (2) is satisfied:
θ₁≤θₜ>θ₂ (2),
where θₜ is an male between a lead-out direction of the treatment tool and the distal end direction of the longitudinal axis direction when the elevator is in the forward position.

5. The endoscope according to claim 3,
wherein the angle θ₂ and an angle θ_{gw} satisfy the following expression (3):
θ₂≤θ_{gw} (3),
where θ_{gw} is an angle between a lead-out direction of the guidewire and the distal end direction of the longitudinal axis direction when the elevator is in the elevated position.

6. The endoscope according to claim 5, wherein the angle θ_{gw} is less than or equal to 90°.

7. The endoscope according to claim 1,
wherein the convex surface and the concave surface are each arc-shaped, and
wherein a radius of curvature R₁ of the concave surface is greater than a radius of curvature R₂ of the convex surface.

8. The endoscope according to claim 1, wherein the concave surface is integrally formed with a lead-out port forming wall that forms the treatment tool lead-out port.

9. The endoscope according to claim 1, wherein the treatment tool has a higher bending stiffness than the guidewire.

10. The endoscope according to claim 1,
wherein the distal end body has an ultrasonic transducer having an ultrasonic vibrator, and
wherein the elevator is disposed on the proximal end side in the longitudinal axis direction relative to the ultrasonic transducer.
